(19) United States
(12) Patent Application Publication
(10) Pub. No.: US 2025/0049743 A1
(43) Pub. Date: Feb. 13, 2025

(54) METHOD OF PRODUCING AN ORAL OSMOTIC PHARMACEUTICAL DELIVERY SYSTEM AND A PHARMACEUTICAL BATCH PRODUCED USING THE SAME

(71) Applicant: United Therapeutics Corporation, Silver Spring, MD (US)

(72) Inventors: Courtney Chang, Raleigh, NC (US); Jason Staton, Leasburg, NC (US); Dana Cella, Pfafftown, NC (US); Karen Polowy, Raleigh, NC (US); Heather Pollinger Cato, Pittsboro, NC (US); Mike Camp, Durham, NC (US)

(73) Assignee: United Therapeutics Corporation, Silver Spring, MD (US)

(21) Appl. No.: 18/796,639

(22) Filed: Aug. 7, 2024

Related U.S. Application Data
(60) Provisional application No. 63/531,428, filed on Aug. 8, 2023.

(51) Int. Cl. A61K 31/192; A61K 9/00; A61K 9/28

(52) U.S. Cl. CPC A61K 31/192; A61K 9/0004; A61K 9/2866; A61K 9/2886; A61K 9/2893

(57) ABSTRACT

A method of producing an oral osmotic pharmaceutical delivery system by using a statistical modeling. The method includes determining a desired average dissolution profile for the active pharmaceutical ingredient; and controlling tablet strength, acetyl content of the cellulose acetate and weight gain of the semi-permeable membrane of the tablet to produce a pharmaceutical batch of tablets having the desired dissolution profile.

### CROSS-REFERENCE TO RELATED APPLICATIONS

The present application claims priority to U.S. Provisional Application No. 63/531,428, filed Aug. 8, 2023, the entire contents of which are incorporated herein by reference.

### BACKGROUND

The present disclosure relates generally to the field of manufacture of an extended release oral tablet. More specifically, the present disclosure relates to use of a statistical modeling in the manufacture of an extended release oral tablet that utilizes osmotic delivery of the drug by means of a semipermeable membrane containing cellulose acetate.

Extended release tablets having an osmotically active drug core surrounded by a semi-permeable membrane are known in the art. These osmotic dosage forms function by allowing water from gastric or intestinal fluid, to flow through the semi-permeable membrane and dissolve the active ingredient in the core so it can be released through one or more passageways in the membrane. An elementary osmotic pump (EOP) delivery system requires that the drug is in solution in order to be delivered in a controlled and predictable manner. The drug in solution is pumped out due to the osmotic gradient generated across the semi-permeable membrane.

Solubility of the drug in aqueous media is usually used as a reference to assess whether there is a need for a solubilizer in the core formulation. Several drug delivery platforms were developed to overcome the challenges of limited solubility of poorly soluble medicinal agents. If the drug is insoluble, an elementary osmotic pump system will not function properly. One approach for delivering pharmaceutical agents that are insoluble in aqueous solvents was developed by Kuczynski et al., (U.S. Pat. No. 5,545,413). In their approach, the interior of the tablet or capsule is characterized by two core layers, one containing the pharmaceutical agent (again to be released through openings, or holes, in the wall of the tablet or capsule) and the other being a layer of material that swells when coming into contact with water. The material that swells or expands to an equilibrium state when exposed to water or other biological fluids is referred to as an “osmopolymer”. This volume expansion is used to physically force the pharmaceutical agent out through the openings, which have been formed in the wall, shell or coating during the manufacture. The pharmaceutical agent is primarily released in the form of insoluble particles, which therefore have limited bioavailability. This method has commonly been referred to as the “push/pull” approach. See, for example, U.S. Pat. Nos. 5,422,123; 4,783,337; 4,765,989; 4,612,008; and 4,327,725. The patent literature has taught this approach for delivering adequate doses, at controlled rates and for extended times, of a broad variety of drugs.

Other osmotic delivery systems have also been described. See, for example, U.S. Pat. Nos. 4,609,374; 4,036,228; 4,992,278; 4,160,020; and 4,615,698. The osmopolymers used in these types of systems are components whose function is to swell when they interact with water and aqueous fluids. This swelling effect is defined in these patents as a property of imbibing fluid so as to expand to a very high degree, usually exhibiting a 2- to 50-fold volume increase.

Rudnic et al., (U.S. Pat. Nos. 6,110,498; 6,284,276; 6,361,796, and 6,514,532) used sodium lauryl sulfate and other solubilizers to enhance the solubility of glipizide, a poorly soluble drug, to deliver it from an elementary type of osmotic system in a sustained manner. This system of Rudnic is comprised of (a) a semi-permeable wall that maintains its integrity during pharmaceutical delivery, and which has at least one passage there through; (b) a single, homogeneous composition within said wall, which composition consists essentially of (i) a pharmaceutically active agent, (ii) at least one non-swelling solubilizing agent which enhances the solubility of the pharmaceutically active agent; (iii) at least one non-swelling osmotic agent, and, optionally, (iv) a non-swelling wicking agent dispersed throughout the composition which enhances the surface area contact of the pharmaceutical agent with the incoming aqueous fluid.

Thombre et al., (U.S. Pat. No. 5,697,922) used meglumine as a solubilizing agent for glipizide. This patent suggests coating meglumine with semi-permeable polymeric films to extend the release of the solubilizer from the core. Thombre et al. argued that non-encapsulated solubilizers would leave the core early, leaving the drug behind in an unsolubilized form. This loss of solubilizer results in an erratic release or no release at all. The problem with this approach is that it is very complex, because it involves coating of the solubilizing excipient during the manufacture of the tablet. This process limits its practical significance. Also, the amount of solubilizing excipient used in this approach is exceedingly high. See also, U.S. Pat. No. 5,698,220, which discloses the use of 90% meglumine (aka, N-methylglucamine) in an osmotic dosage form for delivering glipizide.

Prostacyclins are characterized by a very short half-life ranging from several minutes to several hours, which makes sustained oral delivery of this compounds problematic. A chemically stable analog of prostacyclin, treprostinil, has presented problems in sustained oral delivery with the prior art. Although treprostinil sodium (Remodulin®) is approved by the Food and Drug Administration (FDA) for subcutaneous and intravenous administration, treprostinil as the free acid has an absolute oral bioavailability of less than 10%. Though oral preparations of treprostinil have been disclosed (e.g. U.S. Patent Applications 20050165111 and 20050282903, and U.S. Pat. Nos. 5,153,222, 5,028,628, and 6,054,486), none of these publications addresses an issue of an erratic/incomplete release of treprostinil from oral controlled release dosage forms. US {atent Publication 20050282901 to Phares discloses a composition comprising a prostacyclin (treprosinil) and an additional cardiovascular agent that enhances the oral bioavailability of treprostinil.

Kidane et al., (U.S. Pat. No. 8,747,897) disclosed sustained release, high bioavailability preparations having treprostinil as the only active agent, surrounded by a semi-permeable membrane. Kidane et al. also relates to Orenitram® ER Tablets, which are specifically formulated to be released over a 24-hour period after ingestion via an osmotic pump dissolution mechanism. As described in NDA 203496, Pharmaceutical Development of Orenitram®, Section 3.2.P.2, Orenitram® is “a homogenous core tablet that is surrounded by a semipermeable membrane. A wicking agent in the core tablet enhances the contact surface area of the active pharmaceutical ingredient (API) with the gastrointestinal tract fluid. The API is released from the dosage form through a laser-drilled aperture as a solution or suspension due to an osmotic-pressure gradient across the semi-permeable membrane. Therefore, the API is released from the tablet independent of the fluid conditions of the gastrointestinal tract since the driving force from the tablet is an osmotic pressure gradient.”

Despite breadth of prior art directed to extended release tablets having an osmotically active drug core surrounded by a semi-permeable membrane, no teaching has been suggested for manufacturing of a batch of the extended release tablets to achieve uniform character and quality within specified limits.

### SUMMARY

One embodiment relates to a method of producing a pharmaceutical batch of a controlled release pharmaceutical composition wherein the controlled release pharmaceutical composition is a tablet comprising a core comprising an active pharmaceutical ingredient, said core surrounded by a semi-permeable membrane comprising cellulose acetate and an outer coat, the method comprising: determining a desired average dissolution profile for the active pharmaceutical ingredient; and controlling tablet strength, acetyl content of the cellulose acetate and weight gain of the semi-permeable membrane of the tablet to produce a pharmaceutical batch of tablets having the desired dissolution profile.

One embodiment relates to a pharmaceutical batch of a controlled release pharmaceutical composition produced by the same method.

Yet another embodiment is a method of producing a pharmaceutical batch of a controlled release pharmaceutical solid dosage form comprising a core comprising an active pharmaceutical ingredient, said core surrounded by a semi-permeable membrane comprising cellulose acetate, the method comprising: (a) selecting an amount of the active pharmaceutical ingredient for a solid dosage form; (b) producing a batch of cores, each comprising the active pharmaceutical ingredient in the selected amount; (c) obtaining a lot of cellulose acetate; (d) determining a content of acetyl in the obtained lot; (e) determining an effective coating amount based on the selected amount of the active pharmaceutical ingredient and the determined content of acetyl from statistical data for previously produced pharmaceutical batches for the selected amount of the active ingredient; and (f) for each core of the batch of cores, forming around the core a semi-permeable membrane coating using the effective coating amount determined in step (e).

### FIGURES

FIG. 1 shows 6-hour dissolution results as a function of functional coat weight gain.

FIG. 2 shows influence of tablet strength on 6-hour dissolution results at 3.7% w/w functional coat weight gain.

FIG. 3 shows influence of acetyl content (%) of cellulose acetate on 6-hour dissolution results at 3.7% w/w functional coat weight gain.

FIG. 4 shows statistical model predictions of average 6-hour dissolution for 0.125-mg Orientram® ER tablets, wherein black cells indicate combinations for which the average 6-hr prediction is 54-56% LCD and the 95% prediction interval is within specification (center of specification) and grey cells indicate combinations for which the 95% prediction interval lies completely within the 6-hr specification (45-65% LCD).

### DETAILED DESCRIPTION

As used herein, “batch” or “pharmaceutical batch” refers to a “specific quantity of drug or other material that is intended to have uniform character and quality, within specified limits, and is produced according to a single manufacturing order during the same cycle of manufacture.” See 21 C.F.R. 210.3 (b) (2). “Batches” or “pharmaceutical batches” as defined herein may include a single batch, wherein the single batch is representative of all commercial batches (see generally, Manual of Policies and Procedures, Center for Drug Evaluation and Research, MAPP 5225.1, Guidance on the Packaging of Test Batches at 1), and may also include all batches prepared by a same compounding process.

As used herein, “drug product” means any dosage form containing an active ingredient. See 21 C.F.R. 210.3 (b) (7).

As used herein, “active ingredient” means any component that is intended to furnish pharmacological activity or other direct effect in the diagnosis, cure, mitigation, treatment, or prevention of disease, or to affect the structure or any function of the body of man or other animals.” See 21 C.F.R. 210.3 (b) (7).

As used herein, “lot” means “a batch, or a specific identified portion of a batch, having uniform character and quality within specified limits; or, in the case of a drug product produced by continuous process, it is a specific identified amount produced in a unit of time or quantity in a manner that assures its having uniform character and quality within specified limits.” See 21 C.F.R. 210.3 (b) (10).

As used herein, “strength” means “the concentration of drug substance,” (e.g. weight/weight. See 21 C.F.R. 210.3 (b) (16).

As used herein, the terms “complexing agents” and “micelle forming agents” are used herein as described in Chapters 14 and 20, respectively, of the 20th edition of Remington's The Science and Practice of Pharmacy (20th ed., Lippincott, Williams and Williams, 2000).

As used herein, a wicking agent is defined as any material with the ability to draw water into the network of a delivery dosage form. A wicking agent provides enhanced flow channels for the pharmaceutical agent which has been made predominantly into its solubilized form.

The extended release tablet disclosed below is exemplary only. Numerous other variations will be appreciated by those skilled in the art, in view of the disclosure herein. For example, the delivery system described herein is not limited to a particular dosage form such as tablets. The dosage form containing the appropriate release-enhancing agents can be prepared, e.g., in the form of capsules or pellets. The exact compositions, methods of preparation and embodiments shown are not limiting of the invention, and any obvious modifications will be apparent to one skilled in the art.

In generating a statistical model described in this application, an osmotic delivery system with treprostinil (Orenitram® tablets) was used. In one embodiment, treprostinil may be treprostinil diolamine (a.k.a. treprostinil diethanolamine). The osmotic delivery system used herein may include: a) at least one medicinal agent with high aqueous solubility that exhibits limited and/or erratic and unpredictable release when formulated into an osmotic dosage from; (b) at least one release enhancing agent; (c) at least one osmotic agent; (d) optionally, a binder to aid tableting; (e) optionally, at least one lubricant to aid the tableting process; (f) typically at least one coating polymer; and (g) optionally, at least one plasticizer.

In one embodiment, suitable release enhancing agents include wicking agents, such as high HLB surfactants (for example Tween 20, Tween 60 or Tween 80; ethylene oxide propylene oxide block copolymers (a.k.a. Pluronics®),), ionic surfactants such as sodium lauryl sulfate, sodium docusate, non-swelling hydrophilic polymers such as cellulose ethers, and polyethylene glycols (PEGs); complexing agents such as: polyvinyl pyrrolidone, cyclodextrins and non-ionic surface active agents; and micelle forming agents, which may be surface active agents such as Tweens-(Poly(ethylene Oxide) modified sorbitan monoesters), Spans (fatty acid sorbitan esters), sodium lauryl sulfate, and sodium docusate.

The release enhancing agents are incorporated in the core tablet formula and constitute from 0.5% to 90% by weight of the formula, preferably from 1% to 50%. Most preferably, release-enhancing agents constitute from 1% to 20% by weight of the formulation. They can be incorporated during granulation or post-granulation. The release enhancing agent(s) can be added in the form of solid powder or can be dissolved in the granulating liquid and sprayed during granulation. In order to achieve intimate mixing with the drug(s) the release enhancing agent can be mixed with the drug alone before the other excipients are incorporated. Alternatively, the release enhancing agent(s) and the drug can be dissolved in the granulating liquid and sprayed during granulation. In yet another way, the release enhancing agent(s) and the drug can be dissolved in a solvent, and when the solution is dried, the solid mass thus obtained can be milled and then mixed with the rest of the excipients for further processing.

Osmotic agents are well known to those skilled in the art. Osmotic agents may be simple sugars such as sucrose, xylitol, glucose, lactose; salts such as sodium chloride, potassium chloride; low molecular weight hydrophilic polymers such as cellulose ethers, maltodextrins, and cyclodextrins. Osmotic agents can be incorporated in the amount of from 1% by weight to 90% by weight, preferably from 50% to 90% and most preferably from 80% to 90% by weight. Osmotic agents are typically incorporated in the formula during granulation.

The granules can also be blended with other excipients as needed to aid the manufacturing of the desired dosage form: tablets, capsules or pellets. Tablets are compressed on a standard rotary tablet press.

The core tablet is typically coated with a semi-permeable membrane containing at least one plasticizer. The coating polymer is dissolved with at least one plasticizer in an appropriate solvent or a mixture of solvents and sprayed on the tablets for coating. The coating polymers include, but are not limited, to cellulose acetate. The coating membrane preferably also contains at least one plasticizer to improve flexibility and durability of the coat. Such plasticizers include, but are not limited to, triethyl citrate (TEC), propylene glycol (PG), or mixtures thereof in ratios of TEC to PG ranging from 25:75 to 75:25; Tween 80, polyethylene glycols (PEGs); other polyoxyethylene sorbitan esters, triacetin, diethyl phthalate, mineral oil, tributyl sebacate, and glycerol. The coating level can vary from 1% to 25%, preferably from 2% to 20%, and most preferably from 3% to 10% by weight. In one embodiment, the functional coat is a solution of cellulose acetate and triethyl citrate in acetone that is applied in a pan coater.

The semi-permeable membrane also includes at least one opening to provide for the osmotic delivery of the drug(s). In general the at least one opening has a diameter of from 50 μm to 1000 μm, preferably from 100 μm to 800 μm. The opening is formed by drilling using a laser or any other appropriate hole drilling system. The opening can be of any shape. The various shapes contemplated for this invention include, but are not limited to round, cross-shaped, rectangular, diamond, star, and square shapes. As the dosage unit (osmotic tablet) imbibes water, the release-enhancing agents go into solution providing an environment for the drug to dissolve. The osmotic agent(s) in the core tablet draws water into the core tablet creating an osmotic gradient across the semi-permeable membrane. The osmotic gradient pushes the drug in the solution out through the laser-drilled hole.

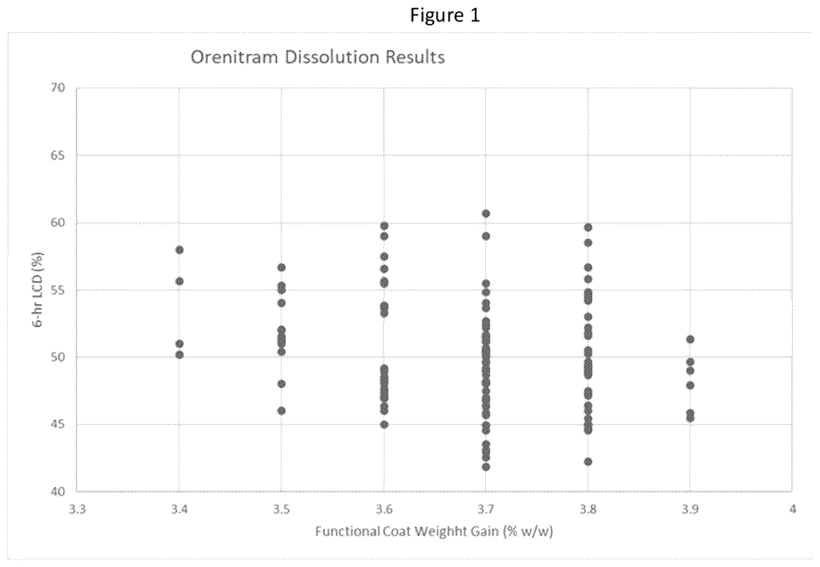

For an osmotic pump tablet, the rate of dissolution is known to be dependent on the functional coat weight gain, which is a surrogate for the thickness of the semipermeable membrane. As functional coat weight gain remains the largest factor in the dissolution results, the tablet strength and acetyl content influences can only be seen graphically when holding functional coat weight gain constant. Referring to FIG. 1, FIG. 1 shows Orenitram® 6-hr dissolution results as a function of functional coat weight gain (% w/w). The overall trend of higher coating weight gain, indicating a thicker semi-permeable membrane layer, resulting in slower release of the active ingredient from the tablet is apparent.

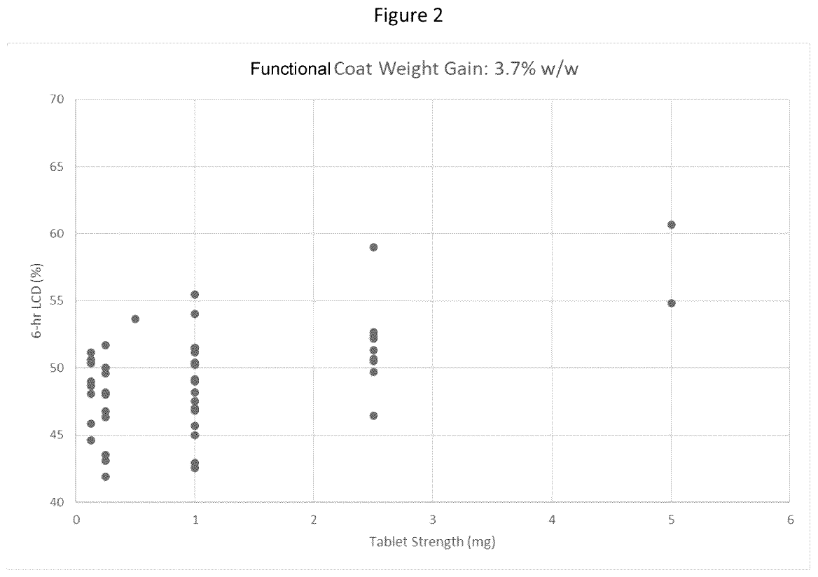

FIG. 2 shows influence of the tablet strength on the 6-hr dissolution results at a given functional coat weight gain. Specifically, the influence of tablet strength on 6-hour dissolution results can be seen at 3.7% w/w functional coat weight gain, which was the most commonly achieved weight gain. As can be seen in FIG. 2, tablets containing higher amounts of active ingredient exhibited a faster rate of dissolution rate because a higher quantity of active ingredients results in a higher osmotic pressure.

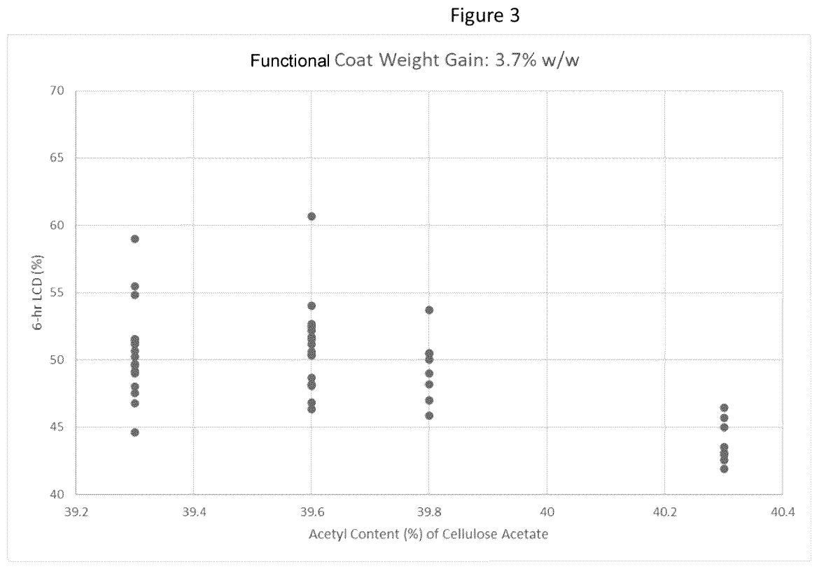

In addition, it was determined that the acetyl content (%) of the cellulose acetate impacts the dissolution rate of this extended release tablet. FIG. 3 shows influence of the acetyl content (%) of the cellulose acetate on the 6-hr dissolution results at a given functional coat weight gain, for example, at 3.7 w/w functional coat weight gain. Tablets coated with cellulose acetate with higher acetyl contents exhibited a slower dissolution rate because the permeability to gas and moisture of cellulose acetate decreases, thereby decreasing the drug dissolution rate.

In view of the above findings, tablet strength, functional coat weight gain, and acetyl content (%) data for historical batches of Orenitram® and the corresponding dissolution results at release testing were used to generate the statistical model. In one embodiment, the amount of active ingredient may range from 0.125-mg to 5-mg per tablet. In one embodiment, the acetyl content of cellulose acetate of the semi-permeable membrane may range from 39.3% to 40.3%. In one embodiment, the desired functional weight gain of the semi-permeable membrane may range from 3.2% w/w to 4.1% w/w.

Specifically, a linear regression model was generated to predict the average 6-hr dissolution result with tablet strength as a categorical variable and functional coat weight gain and cellulose acetate acetyl content as covariates. While a linear regression model was selected for its simplicity and applicability into a manufacturing setting, non-linear regression models considered suitable by a person of ordinary skill in the art may also be generated. As an example, the linear regression model was based on results collected from 131 batches of Orenitram® tablets.

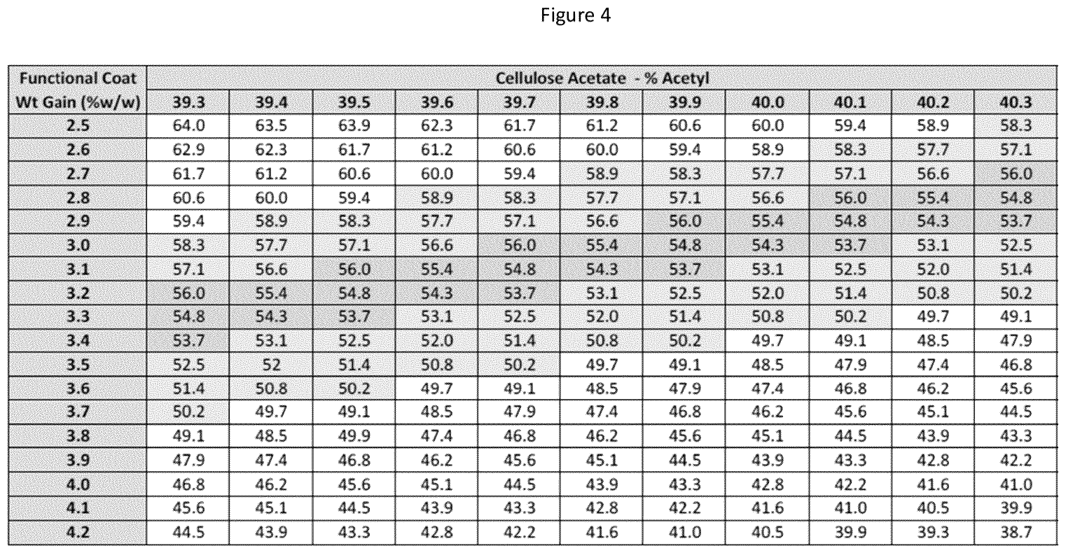

As an example, FIG. 4 shows a prediction table generated based on the linear regression model for each combination of functional coat weight gain (within the range allowed by the drug product NDA) and acetyl content for the cellulose acetate (within the range allowed by the vendor specification). The prediction table of FIG. 4 is based on the 0.125-mg tablet strength. For each combination of functional coat weight gain (within the range allowed by the drug product NDA) and acetyl content for the cellulose acetate (within the range allowed by the vendor specification), a 6-hr average dissolution result was determined. For each tablet batch to be functional coated, with the tablet strength determined and the cellulose acetate lot assigned, the target functional coat weight gain was selected to target the center of the 6-hr dissolution specification using the statistical model prediction tables. In other words, the target functional coat weight gain was adjusted based on the acetyl content (%) of the cellulose acetate lot to meet the 6-hr dissolution specification. A person of ordinary skill in the art would easily understand that because table strength also influences the dissolution rate, the prediction table would be different for a different tablet strength.

As disclosed herein, the adjustment of the target weight gain based on the statistical model predictions may be achieved in by determining a desired strength of tablets to be manufactured, assigning lot of cellulose acetate to the tablet batch, selecting the target functional coat weight gain for the tablet batch based on the statistical model. Such use of the statistical model reduces the percentage of the produced pharmaceutical batches that fail to meet the desired average dissolution rate. For example, as can be seen in Table 1 below, use of the statistical model achieves improved manufacturing result, yielding no batches that fail to meet a desired specification compared to 4.2% failure when manufactured without use of the statistical modeling.

TABLE 1
Comparison of pharmaceutical batches meeting the desired
average dissolution result produced by a process
using and without using a statistical model.

| | Process | New Process |
|---|---|---|
| | (No Model) | (With Model) |
| Total Batches Manufactured | 142 | 26 |
| Batches Failing to Meet Spec (OOS) | 6 (4.2%) | 0 |
| Meeting Spec at L1 Testing | 31 (21.8%) | 20 (76.9%) |
| Meeting Spec at L2 Testing | 101 (71.1%) | 6 (23.1%) |
| Meeting Spec at L3 Testing | 4 (2.8%) | N/A |

According to some embodiments, there is a method of producing a pharmaceutical batch of a controlled release pharmaceutical solid dosage form comprising a core comprising an active pharmaceutical ingredient, said core surrounded by a semi-permeable membrane comprising cellulose acetate, the method comprising: (a) selecting an amount of the active pharmaceutical ingredient for a solid dosage form; (b) producing a batch of cores, each comprising the active pharmaceutical ingredient in the selected amount; (c) obtaining a lot of cellulose acetate; (d) determining a content of acetyl in the obtained lot; (e) determining an effective coating amount based on the selected amount of the active pharmaceutical ingredient and the determined content of acetyl from statistical data for previously produced pharmaceutical batches for the selected amount of the active ingredient; and (f) for each core of the batch of cores, forming around the core a semi-permeable membrane coating using the effective coating amount determined in step (e).

The active pharmaceutical ingredient may be a water soluble pharmaceutical agent having water solubility of at least 30 mg/ml or at least 50 mg/ml or at least 70 mg/ml or at least 100 mg/ml.

In some embodiments, the active pharmaceutical ingredient may treprostinil or its pharmaceutically acceptable salt.

In some embodiments, the active pharmaceutical ingredient may be a water soluble salt of treprostinil having water solubility of at least 30 mg/ml or at least 50 mg/ml or at least 70 mg/ml or at least 100 mg/ml. For example, in some embodiments, the a water soluble salt of treprostinil is treprostinil sodium or treprostinil diethanolamine.

In some embodiments, selecting the amount of the active pharmaceutical ingredient may involve selecting an amount of treprostinil salt, such as treprostinil diethanolamine, which amount may be equivalent to an amount of treprostinil as free acid from 0.125 mg to 5 mg. For example, selecting the amount of the active pharmaceutical ingredient may involve selecting an amount of treprostinil salt, such as treprostinil diethanolamine, which amount may be equivalent to an amount of treprostinil as free acid selected from 0.125 mg (equivalent to 0.159 mg of treprostinil diethanolamine); 0.25 mg (equivalent to 0.317 mg of treprostinil diethanolamine); 1 mg (equivalent to 1.27 mg of treprostinil diethanolamine); 2.5 mg (equivalent to 3.17 mg of treprostinil diethanolamine) and 5 mg (equivalent to 6.35 mg of treprostinil diethanolamine).

Besides the active pharmaceutical agent, such as treprostinil salt, e.g. treprostinil diethanolamine, the core may comprise additional ingredients, such as a release enhancing agent, such as sodium lauryl phosphate, an osmotic agent such as sucrose, xylitol, glucose, lactose, sodium chloride, potassium chloride, cellulose ethers, maltodextrins, and cyclodextrins; and a lubricant, such as magnesium stearate.

The lot of cellulose acetate may be obtained from a commercially available source. Lots of cellulose acetate from commercially available sources may have a distribution of acetyl content within the manufacturer's specification. For example, in some embodiments, lots of cellulose acetate may have a content of acetyl from 29.0% to 44.8% or from 33% to 42% or from 36% to 41% or from 39.3% to 40.3%. The present method involves determining the specific acetyl content for the obtained lot of acetyl acetate within the distribution of acetyl content.

The effective coat amount may be determined based on the selected amount of the active pharmaceutical ingredient and the determined acetyl content in the specific (obtained) lot of cellulose acetate based on statistical data of previously produced pharmaceutical batches for the selected amount of the same active pharmaceutical agent produced using a range of cellulose acetate amounts for cellulose acetate lots with various acetyl contents. Preferably, the determined acetyl content is within the range of acetyl contents of the cellulose acetate lots used to produce the previously produced batches. Based on the statistical data for the previously produced pharmaceutical batches, the effective coat amount may be selected as an amount of cellulose acetate used for preparing a semi-permeable membrane, which provided a statistically significant better test result for the determined acetyl content in the obtained lot of cellulose acetate. The test result may be, for example, a 6 hour dissolution test.

In some embodiments, the statistical data may be presented a table similar to the one in FIG. 4. FIG. 4 presents horizontally the range of acetyl contents of the cellulose acetate lots used to produce the previously produced batches of 0.125 mg orenitram tablets and vertically the range of cellulose acetate amounts (functional coat wt gain) in semi-permeable membranes of the previously produced batches of 0.125 mg orenitram tablets. The boxes in FIG. 4 present the results of 6 hour dissolution test for previously produced batches of 0.125 mg orenitram tablets. The darker (green) cells indicate combinations for which the average 6-hr prediction is 54-56% LCD and the 95% prediction interval is within specification (center of specification). The lighter (orange) cells indicate combinations for which the 95% prediction interval lies completely within the 6-hr specification (45-65% LCD).

Based on the table in FIG. 4, for 0.125 mg orenitram tablet, for the determined 40.3% acetyl content in the obtained cellulose acetate lot, an amount of cellulose acetate for preparing a semipermeable membrane is determined to be 2.5% to 3.2%, preferably 2.7% to 2.9%, while for determined 39.3% acetyl content in the obtained cellulose acetate lot, an amount of cellulose acetate for preparing a semipermeable membrane is determined to be 3.0% to 3.7%, preferably 3.2% to 3.4%.

Use of the statistical data for the previously produced batches while determining an amount of cellulose acetate in a semi-permeable membrane of a new batch may decrease a risk that the new batch will fail a quality test compared to a batch which is produced without determining an amount of cellulose acetate in a semi-permeable membrane using of the statistical data for the previously produced batches.

Beside the cellulose acetate, the semi-permeable membrane may include a plasticizer, such as triethyl citrate.

In some embodiments, the solid dosage form may be a tablet. In some embodiments, the tablet may be an Orenitram™ tablet (controlled release formulation of Treprostinil diethanolamine).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all parts and percentages are set forth by weight, unless otherwise indicated.

The entire disclosures of all applications, patents and publications cited herein are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed is:

1. A method of producing a pharmaceutical batch of a controlled release pharmaceutical composition, wherein the controlled release pharmaceutical composition is a tablet comprising a core comprising an active pharmaceutical ingredient, said core surrounded by a semi-permeable membrane comprising cellulose acetate and an outer coat, the method comprising:
determining a desired average dissolution profile for the active pharmaceutical ingredient; and
controlling tablet strength, acetyl content of the cellulose acetate and weight gain of the semi-permeable membrane of the tablet to produce a pharmaceutical batch of tablets having the desired dissolution profile.

2. The method of claim 1, wherein the desired average dissolution result is determined at 6 hours.

3. The method of claim 1, wherein the amount of active pharmaceutical ingredient ranges from 0.125-mg to 5-mg per tablet.

4. The method of claim 3, wherein the active pharmaceutical ingredient is treprostinil diethanolamine.

5. The method of claim 1, wherein the acetyl content ranges from 39.3 to 40.3%.

6. The method of claim 1, wherein the desired weight gain of the semi-permeable membrane ranges from 3.2 to 4.1% w/w.

7. The method of claim 1, wherein a percentage of the pharmaceutical batches not meeting the desired average dissolution result is less than 4.2%.

8. A pharmaceutical batch produced by the method of claim 1.

9. The pharmaceutical batch of claim 8, wherein the amount of active pharmaceutical ingredient ranges from 0.125-mg to 5-mg per tablet.

10. The pharmaceutical batch of claim 8, wherein the acetyl content ranges from 39.3 to 40.3%.

11. The pharmaceutical batch of claim 10, wherein the active pharmaceutical ingredient is treprostinil diethanolamine.

12. The pharmaceutical batch of claim 11, wherein the semi-permeable membrane comprises one laser-drilled opening suitable for osmotic delivery.

13. The pharmaceutical batch of claim 12, wherein the tablet further comprises A) at least one release enhancing agent selected from a group consisting of wicking agents, complexing agents, and micelle-forming agents, wherein
i) the wicking agents are selected from the group consisting of high HLB surfactants, ionic surfactants, and non-swelling hydrophilic polymers,
ii) the complexing agents are selected from the group consisting of polyvinyl pyrrolidone, cyclodextrins, and non-ionic surface active agents, and
iii) the micelle-forming agents are selected from the group consisting of poly(ethylene oxide) modified sorbitan monoesters, fatty acid sorbitan esters, sodium lauryl sulfate, and sodium docusate.

14. A method of producing a pharmaceutical batch of a controlled release pharmaceutical solid dosage form comprising a core comprising an active pharmaceutical ingredient, said core surrounded by a semi-permeable membrane comprising cellulose acetate, the method comprising: (a) selecting an amount of the active pharmaceutical ingredient for a solid dosage form; (b) producing a batch of cores, each comprising the active pharmaceutical ingredient in the selected amount; (c) obtaining a lot of cellulose acetate; (d) determining a content of acetyl in the obtained lot; (e) determining an effective coating amount based on the selected amount of the active pharmaceutical ingredient and the determined content of acetyl from statistical data for previously produced pharmaceutical batches for the selected amount of the active ingredient; and (f) for each core of the batch of cores, forming around the core a semi-permeable membrane coating using the effective coating amount determined in step (e).

15. The method of claim 14, wherein the active pharmaceutical ingredient is treprostinil or a salt thereof.

16. The method of claim 15, wherein the active pharmaceutical ingredient is a salt of treprostinil.

17. The method of claim 16, wherein the salt of treprostinil is treprostinil diethanolamine.

18. The method of claim 17, wherein said selecting comprises selecting an amount of treprostinil diethanolamine corresponding to an amount of treprostinil from 0.125 mg to 5 mg.

19. The method of claim 18, wherein said selecting comprising selecting an amount of treprostinil diethanolamine corresponding to an amount of treprostinil selected from the group consisting of 0.125 mg, 0.25 mg, 1 mg, 2.5 mg and 5 mg.

20. The method of claim 14, wherein the determined content in the obtained lot is from 39.3% to 40.3%.

21. The method of claim 15, wherein each core of said produced batch of cores further comprises a release enhancing agent.

22. The method of claim 21, wherein the release enhancing agent is sodium lauryl sulfate.

23. The method of claim 21, wherein each core of said produced batch of cores further comprises xylitol, maltodextrin and magnesium stearate.

24. The method of claim 15, wherein the semi-permeable membrane further comprises triethyl citrate.

25. The method of claim 14, wherein the solid dosage form is a tablet.

26. A pharmaceutical batch produced according to the method of claim 14.
